(19) United States
(12) Patent Application Publication
(10) Pub. No.: US 2025/0058297 A1
(43) Pub. Date: Feb. 20, 2025

(54) Zirconium-Based Metal-Organic Framework Material and Preparation Method Therefor and Use Thereof, and Adsorption Separation Device and Method

(71) Applicant: PetroChina Company Limited, Beijing (CN)

(72) Inventors: Qihan GONG, Beijing (CN); Hao WANG, Beijing (CN); An MA, Beijing (CN); Jiayu DING, Beijing (CN); Shang ZHANG, Beijing (CN); Liang YU, Beijing (CN); Guangjuan GUO, Beijing (CN); Yawen BO, Beijing (CN); Hongyue YU, Beijing (CN); Kebin CHI, Beijing (CN)

(21) Appl. No.: 18/725,326

(22) PCT Filed: Dec. 2, 2022
(86) PCT No.: PCT/CN2022/136094

§ 371 Date: Jul. 9, 2024

(30) Foreign Application Priority Data
Dec. 31, 2021 (CN) 202111675243.1

(51) Int. Cl. B01J 20/22; B01J 20/30; B01J 20/34; C07C 7/13; C07F 7/00

(52) U.S. Cl. CPC B01J 20/226; B01J 20/3078; B01J 20/3425; B01J 20/3458; B01J 20/3483; C07C 7/13; C07F 7/003

(57) ABSTRACT

The present invention provides a zirconium-based metal-organic framework material and a preparation method therefor and the use thereof, and an adsorption separation device and method. The zirconium-based metal-organic framework material has a chemical structural formula of [C₁₈H₆O₁₆Zr₃]ₙ, and comprises zirconium and an organic ligand forming a coordination bond with zirconium, wherein the organic ligand is diphenylethyne-3,3′,5,5′-tetracarboxylic acid. The molecular structure of the zirconium-based metal-organic framework material of the present invention is a three-position network structure having a one-dimensional channel; and in the present invention, the size of the one-dimensional channel is accurately controlled by changing the aspect ratio of the organic ligand, such that the zirconium-based metal-organic framework material efficiently separates a hexane isomer by means of a kinetic effect.

### CROSS-REFERENCE TO RELATED APPLICATIONS

This application is a continuation of International Application No. PCT/CN2022/136094, filed on Dec. 2, 2022, which claims priority to Chinese Patent Application No. 202111675243.1, filed on Dec. 31, 2021, both of which are hereby incorporated by reference in their entireties.

### TECHNICAL FIELD

The present disclosure relates to the field of adsorption separation technology, and specifically relates to a zirconium-based metal-organic framework material and its preparation method and use, and an adsorption separation device and method.

### BACKGROUND OF ART

The chemical separation process in industry is closely related to the national economy and social development, and is an indispensable and important engine to promote the modernization and development of mankind. However, energy crisis and environmental pollution are two major problems facing the world today. At present, the chemical separation mainly adopts heat-driven separation technology (e.g., distillation), and the energy consumption related to the chemical separation process accounts for about 50% of the industrial energy consumption and 10-15% of the world's total energy consumption, and this process releases large amounts of carbon dioxide and other hazardous gases, which have a serious impact on the environment. Therefore, it is urgent to develop energy-saving and environmentally friendly alternative technologies to reduce the energy consumption required for the chemical separation process in the chemical industry, to decrease the release of hazardous gases and to reduce the environmental pollution.

High octane number gasoline is an important energy substance in current society because of its excellent resistance to detonating. Alkane isomers are one of the main components of gasoline, and the separation of alkane (mainly pentane and hexane) isomers is an indispensable process for the preparation of high octane number gasoline in the petrochemical industry. In the petroleum refining process, pentane and hexane isomers with different degrees of branching are produced by the catalytic isomerization reaction, and separated. The branched isomers with a low octane number (e.g., n-hexane with an octane number of 30) are returned to the catalytic isomerization reactor for recycling, while the branched isomers with a higher octane number (e.g., 2,2-dimethylbutane with an octane number of 92) can be used as gasoline feedstock. At present, the separation of alkane isomers is generally done by distillation in industry, but the boiling points of alkane isomers are very close to each other, making the distillation separation process complicated, with huge energy consumption and high capital investment.

In order to reduce the energy consumption required for separation and lower the cost, there is an urgent need to develop more efficient, energy-saving and environmentally friendly separation technologies. In recent years, a great many of explorations have been carried out in the process of separating alkane isomers with different branching degrees by adsorption separation technology using solid porous materials as the adsorbent to improve the octane number of gasoline components in many countries, and proved to be practicable. However, the difficulty in efficient separation of hexane isomers, especially mono-branched and di-branched hexanes, has not been well solved so far, and the separation process is complicated and requires a large amount of energy.

### SUMMARY OF INVENTION

In order to solve the above technical problems, a first object of the present disclosure is to provide a zirconium-based metal-organic framework material and a preparation method thereof to solve the problem that the existing separation techniques are unable to separate a mixture of hexane isomers; a second object of the present disclosure is to provide an adsorption separation device and an adsorption separation method to solve the problem that a mixture of hydrocarbons cannot be efficiently separated in the prior art.

In order to achieve the above objects, one aspect of the present disclosure provides a zirconium-based metal-organic framework material (Zr-dpetc) having a chemical structural formula [C₁₈H₆O₁₆Zr₃]ₙ, wherein the zirconium-based metal-organic framework material comprises a zirconium element and an organic ligand which forms a coordination bond with the zirconium element, and the organic ligand is diphenylethyne-3,3′,5,5′-tetracarboxylic acid (abbreviated as dpetc) having the following structural formula:

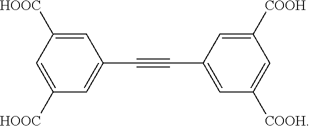 [SMILES: O=C(O)c1cc(C#Cc2cc(C(=O)O)cc(C(=O)O)c2)cc(C(=O)O)c1]

According to a specific embodiment of the present disclosure, preferably, the crystal structure of the zirconium-based metal-organic framework material is as shown in FIGS. 1a and 1b, and the crystal of the zirconium-based metal-organic framework material belongs to the tetragonal crystal system with 14/mmm space group.

The present disclosure is to finely tune the pore shape and size of the metal-organic framework materials by ligand screening under the guidance of topology, thereby achieving high adsorption separation performance for hydrocarbon mixtures. The tetravalent metal ions (e.g., Zr⁴⁺) used in the present disclosure have small radii and high charges, very strong polarization capabilities, and form coordination bonds, containing large covalent components, with oxygen-containing ligands (e.g., carboxylic acids), resulting in a very strong and chemically and thermally stable structure.

According to a specific embodiment of the present disclosure, preferably, the molecular structure of the zirconium-based metal-organic framework material is a three-dimensional network structure with one-dimensional channels.

According to a specific embodiment of the present disclosure, preferably, the one-dimensional channels in the zirconium-based metal-organic framework material have a size of 5-7 Å.

According to a specific embodiment of the present disclosure, preferably, in the zirconium-based metal-organic framework material, the three-dimensional network structure is formed by ZrO₆ octahedron connected with the organic ligand.

According to a specific embodiment of the present disclosure, preferably, in the zirconium-based metal-organic framework material, the ZrO₆ octahedron comprises six Zr atoms.

The present disclosure is to finely tune the pore shape and size of the metal-organic framework materials by ligand screening under the guidance of topology, thereby achieving high adsorption separation performance for hydrocarbon mixtures. The tetravalent metal ions (e.g., Zr⁴⁺) used in the present disclosure have small radii and high charges, very strong polarization capabilities, and form coordination bonds, containing large covalent components, with oxygen-containing ligands (e.g., carboxylic acids), resulting in a very strong and chemically and thermally stable structure. The zirconium-based metal-organic framework material of the present disclosure has a three-dimensional network structure with one-dimensional channels formed by ZrO₆ octahedron formed from six Zr atoms and connected to the organic ligand (dpetc), and the one-dimensional channels have a size of about 7 Å, which allows straight, mono-branched and di-branched hexanes to enter into the channels while forming a certain steric hindrance to inhibit the diffusion of large molecules.

The zirconium-based metal-organic framework material (Zr-dpetc) of the present disclosure is based on a three-dimensional network structure of 8-connected Zr₆ bridged by 4-connected dpetc⁴⁻ organic ligands with scu topology, and the one-dimensional channel has an aperture of about 5-7 Å. The material is formed by connecting six metal centers into octahedron of Zr₆O₄(OH)₄ metallic oxygen clusters via _{μ3}-O and _{μ3}-OH. The octahedron has 12 external connection points and can be connected to up to 12 carboxyl groups via coordination bonds to form ftw topology. However, the pore structure of the material can be modulated by the design of the geometrical configuration of the ligand. In the present disclosure, the pore structure is changed from cage-like pores of ftw topology to one-dimensional channels of scu topology by changing the aspect ratio of the organic ligand. This design idea has mainly the following effects on the separation of alkane isomers: (1) the cage-like pores, due to the small window size, will form a limitation on the diffusion of alkane molecules among the pores, resulting in a large influence of mass transfer during the separation process, which in turn affects the separation efficiency; (2) the pore size is effectively enlarged by changing the cage-like pores into one-dimensional channels, and the final pore size of the material is optimized to be 5-7 Å by controlling the ligand size. Due to the suitable pore size, the thermodynamic selectivity of the material for mono-branched/di-branched alkanes is high. Meanwhile, due to the pore size close to the size of the di-branched isomers, the diffusion of the di-branched alkanes is further limited, thus further improving the separation performance of the material. This is the first time that the adsorption separation performance of alkane isomers is optimized by dual regulation of pore structure and pore size under the guidance of topology.

According to a specific embodiment of the present disclosure, preferably, the zirconium-based metal-organic framework material has a specific surface area of 500-1000 m²/g.

According to a specific embodiment of the present disclosure, preferably, the zirconium-based metal-organic framework material has a thermal decomposition temperature of 350-500° C.

According to a specific embodiment of the present disclosure, preferably, the zirconium-based metal-organic framework material is in the form of white powder crystals.

The zirconium-based metal-organic framework material of the present disclosure has an excellent stability, with a decomposition temperature close to 500° C. No matter whether it is exposed to a high temperature of 120° C. for 7 days, or exposed to air with a relative humidity of up to 90% for 7 days, or placed in hot water at 80° C. for 7 days, it can still maintain the structural integrity without significant decrease in adsorption separation performance.

The zirconium-based metal-organic framework material of the present disclosure can be used as an adsorbent material with the characteristics such as purity, absence of impurities, and regular morphology, and can be used for the kinetic separation of straight, mono-branched, and di-branched isomers of hexane.

A second aspect of the present disclosure provides a method for preparing the zirconium-based metal-organic framework material, comprising the steps of:

mixing a zirconium salt, an organic ligand, a first solvent and an acid in a proportion, and performing a solvothermal reaction or a microwave synthesis process to obtain a semi-finished product; and then removing the solvent in the channels of the semi-finished product, to obtain a finished product of the zirconium-based metal-organic framework material.

According to a specific embodiment of the present disclosure, preferably, the method further comprises washing and drying the semi-finished product before removing the solvent in the channels of the semi-finished product. Preferably, the washing is carried out with a first solvent and the semi-finished product is dried after suction filtration.

According to a specific embodiment of the present disclosure, preferably, the method comprises the steps of:
(1) mixing the zirconium salt, the organic ligand dpetc, the first solvent and the acid in a proportion, dissolving them by ultrasonication or stirring, and feeding into a reactor or glass bottle or another closed container for solvothermal reaction or microwave synthesis;
(2) after the solvothermal reaction or microwave synthesis is finished, washing with the first solvent several times, followed by suction filtration and drying, to obtain the semi-finished product;
(3) removing the reaction solvent molecules in the channels of the semi-finished product structure by vacuum drying or by solvent exchange and then vacuum drying, to obtain the finished product of the zirconium-based metal-organic framework material.

According to a specific embodiment of the present disclosure, preferably, in the method, the molar ratio of the zirconium salt, the organic ligand, the first solvent and the acid is 10: (1-100): (1-100): (2-200).

According to a specific embodiment of the present disclosure, preferably, in the method, the zirconium salt is selected from at least one of zirconium nitrate, zirconium chloride, aluminum zirconium oxide, and zirconium sulfate.

According to a specific embodiment of the present disclosure, preferably, in the method, the organic ligand is diphenylethyne-3,3′,5,5′-tetracarboxylic acid.

According to a specific embodiment of the present disclosure, preferably, in the method, the first solvent is selected from at least one of N,N-dimethylformamide (DMF), N,N-dimethylacetamide (DMA), and N,N-diethylformamide (DEF).

According to a specific embodiment of the present disclosure, preferably, in the method, the acid is selected from at least one of formic acid, acetic acid, hydrochloric acid, and benzoic acid.

According to a specific embodiment of the present disclosure, preferably, when the solvothermal reaction is performed in the method, the reaction temperature of the solvothermal reaction is 80-200° C. and the reaction time is 12-72 h.

Preferably, according to a specific embodiment of the present disclosure, when the microwave synthesis process is performed in the method, the reaction temperature of the microwave synthesis process is 80-180° C. and the reaction time is 1-60 min.

According to a specific embodiment of the present disclosure, preferably, in the method, the removing the solvent in the channels of the semi-finished product is carried out by vacuum drying the semi-finished product, or by impregnating the semi-finished product in a second solvent for solvent exchange, and then vacuum drying.

According to a specific embodiment of the present disclosure, preferably, in the method, the second solvent is selected from at least one of methanol, dichloromethane, ethanol, and acetone.

The method for preparing the zirconium-based metal-organic framework material of the present disclosure has good reproducibility, and the adsorbents are obtained by simple synthesis processes using inexpensive raw materials under mild conditions, and the products are pure and can be prepared rapidly on a large scale.

A third aspect of the present disclosure provides use of the zirconium-based metal-organic framework material in separation of alkane isomers.

According to a specific embodiment of the present disclosure, preferably, in the use, the zirconium-based metal-organic framework material serves as an adsorbent.

According to a specific embodiment of the present disclosure, preferably, in the use, the alkane isomers are hexane isomers.

According to a specific embodiment of the present disclosure, preferably, in the use, the zirconium-based metal-organic framework material adsorbs the hexane isomers in the following order of preference: n-hexane, mono-branched hexane, di-branched hexane.

High octane number gasoline is an important energy substance in current society because of its excellent resistance to detonating. Alkane isomers are one of the main components of gasoline, and the kinetic radii and octane numbers of alkane (mainly pentane and hexane) isomers are shown in Table 1 below:

TABLE 1
Comparison of kinetic radii and octane numbers of hexane isomers

| hexane isomers | n-hexane | 2-methylpentane | 3-methylpentane | 2,3-dimethylbutane | 2,2-dimethylbutane |
|---|---|---|---|---|---|
| kinetic radius (Å) | 4.3 | 5.5 | 5.5 | 5.8 | 6.2 |
| octane number | 24.8 | 74.5 | 73.8 | 91.8 | 101.7 |

The zirconium-based metal-organic framework material of the present disclosure has a three-dimensional network structure with one-dimensional channels formed by ZrO₆ octahedron formed from six Zr atoms and connected to the organic ligand (dpetc), and the one-dimensional channels have a size of about 5-7 Å, which allows straight, mono-branched and di-branched hexanes to enter into the channels while forming a certain steric hindrance to inhibit the diffusion of large molecules. In addition, since different isomers have different interaction force with the pore wall, in the order of hexane>mono-branched hexane>di-branched hexane, the adsorption tendency of the three types of components is in the order of straight hexane>mono-branched hexane>di-branched hexane, which realizes the separation of straight, mono-branched and di-branched hexane isomers.

A fourth aspect of the present disclosure provides an adsorption separation device for alkane isomers, comprising an adsorbent, wherein the adsorbent is the zirconium-based metal-organic framework material.

According to a specific embodiment of the present disclosure, preferably, the adsorption separation device comprises an adsorption column filled with the adsorbent.

According to a specific embodiment of the present disclosure, preferably, the adsorption mode of the adsorption separation device is selected from any one of fixed-bed gas-phase adsorption, simulated moving-bed adsorption and moving-bed adsorption in terms of operation, preferably fixed-bed gas-phase adsorption.

A fifth aspect of the present disclosure provides an adsorption separation method for alkane isomers, comprising: passing a mixture gas or mixture liquid containing hexane isomers through an adsorption column filled with an adsorbent, collecting the constituent isomers one by one, and desorbing the adsorbent after completion of the collection; wherein the adsorbent is the zirconium-based metal-organic framework material.

According to a specific embodiment of the present disclosure, preferably, in the adsorption separation method, the desorption is carried out by one or more of heating, vacuum treatment, and inert gas purging.

In the present disclosure, the hexane isomers comprise straight hexane (n-hexane), mono-branched hexane (2-methylpentane, 3-methylpentane) and di-branched hexane (2,2-dimethylbutane, 2,3-dimethylbutane), and the mixture gas or mixture liquid comprises 2 to 5 constituent hexane isomers. In the adsorption separation method of the present disclosure, the di-branched hexane passes through the adsorption column preferentially, then the mono-branched hexane passes through the adsorption column, and the straight hexane passes through the adsorption column lastly. The di-branched alkane and mono-branched alkane are collected one by one to obtain a high octane number product with high purity. After the straight alkanes have passed, the straight components adsorbed within the adsorbent can be eluted by heating, vacuum treatment, inert gas purging, or a combination of various desorption methods to obtain a C6 straight gas with high purity. The adsorbent described in the present disclosure can be regenerated after only desorption treatment.

According to a specific embodiment of the present disclosure, preferably, in the adsorption separation method, the adsorption temperature is 0-200° C., more preferably 20-150° C.

According to a specific embodiment of the present disclosure, preferably, in the adsorption separation method, the total pressure of the mixture gas during the adsorption is 0-5 bar, more preferably 0.5-1 bar.

According to a specific embodiment of the present disclosure, preferably, in the adsorption separation method, the desorption temperature is 100-200° C.

According to a specific embodiment of the present disclosure, preferably, in the adsorption separation method, the total pressure of the mixture gas during the desorption is 0.05-1 bar.

According to a specific embodiment of the present disclosure, preferably, in the adsorption separation method, the total amount of the hexane isomers is 70-90% of the total mass of the mixture gas or mixture liquid.

According to a specific embodiment of the present disclosure, preferably, in the adsorption separation method, the mixture gas further comprises one or more of impurity gases such as n-pentane, iso-pentane, oxygen, nitrogen, helium, carbon dioxide, and methane in water vapor.

According to a specific embodiment of the present disclosure, preferably, in the adsorption separation method, the mixture liquid further comprises one or more of n-pentane, iso-pentane, water and the like.

Compared with the currently common method of low-temperature distillation, the adsorption separation method for alkane isomers of the present disclosure has the advantages of low cost, energy-saving and environmental protection, simple operation, and the like, and can achieve an improvement in quality and economic benefits for the petrochemical enterprises in the preparation of the blending components of high octane number gasoline.

Compared with the prior art, the zirconium-based metal-organic framework material and the preparation method and use thereof involved in the present disclosure have the following beneficial effects:
(1) The molecular structure of the zirconium-based metal-organic framework material of the present disclosure is a three-dimensional network structure with one-dimensional channels. In the present disclosure, by changing the aspect ratio of the organic ligand, the size of the one-dimensional channels is precisely controlled, such that the zirconium-based metal-organic framework material realizes efficient separation of hexane isomers through kinetic effect, especially efficient separation of mono-branched hexane and di-branched hexane, so as to obtain high octane number alkane products.
(2) Compared with the current separation materials for hexane isomers, the zirconium-based metal-organic framework material of the present disclosure can obtain straight hexane, mono-branched hexane, and di-branched hexane respectively under mild conditions and through only simple operation steps. The obtained di-branched hexane can be used as an additive component of high octane number gasoline, the high-purity n-hexane can be used for ethylene cracking or solvent, and the mono-branched hexane can be returned to the isomerization reactor for the reaction, thus realizing the full utilization of the oil components.
(3) The zirconium-based metal-organic framework material of the present disclosure is free of impurities, with outstanding advantages such as material purity, regular morphology, good stability under harsh environments and high adsorption selectivity. It has good industrial application prospects and can be used in the petrochemical industry for the preparation of high octane number gasoline.

### BRIEF DESCRIPTION OF DRAWINGS

FIG. 1a is a schematic diagram of the spatial structure of the crystal of Zr-dpetc, a zirconium-based metal-organic framework material of the present disclosure;

FIG. 1b is a schematic diagram of the planar structure of the crystal of Zr-dpetc, a zirconium-based metal-organic framework material of the present disclosure;

FIG. 2 is X-ray diffraction patterns of Zr-dpetc samples A-F obtained from Example 1 of the present disclosure after stability testing;

FIG. 3 is thermogravimetric curves of Zr-dpetc samples A and B obtained in Example 1;

FIG. 4 is the nitrogen adsorption-desorption isotherm of Zr-dpetc sample C obtained in Example 1 at 77 K;

FIG. 5 is the adsorption isotherm of Zr-dpetc sample C obtained in Example 1 for n-hexane (nHEX), 3-methylpentane (3MP), and 2,2-dimethylbutane (22DMB) at 30° C.;

FIG. 6 is the adsorption kinetic curve of Zr-dpetc sample C obtained in Example 1 for n-hexane (nHEX), 3-methylpentane (3MP), and 2,2-dimethylbutane (22DMB) at 30° C.;

FIG. 7 is the multicomponent breakthrough curve of Zr-dpetc sample C obtained in Example 1 for a ternary mixture of n-hexane (nHEX), 3-methylpentane (3MP), and 2,2-dimethylbutane (22DMB), wherein the RON in the curve indicates the octane value of elution;

FIG. 8 is the multicomponent breakthrough curve of Zr-dpetc sample C obtained in Example 1 for a quinary mixture of n-hexane (HEX), 2-methylpentane (2MP), 3-methylpentane (3MP), 2,2-dimethylbutane (22DMB) and 2,3-dimethylbutane (23DMB), wherein the RON in the curve indicates the octane value of elution.

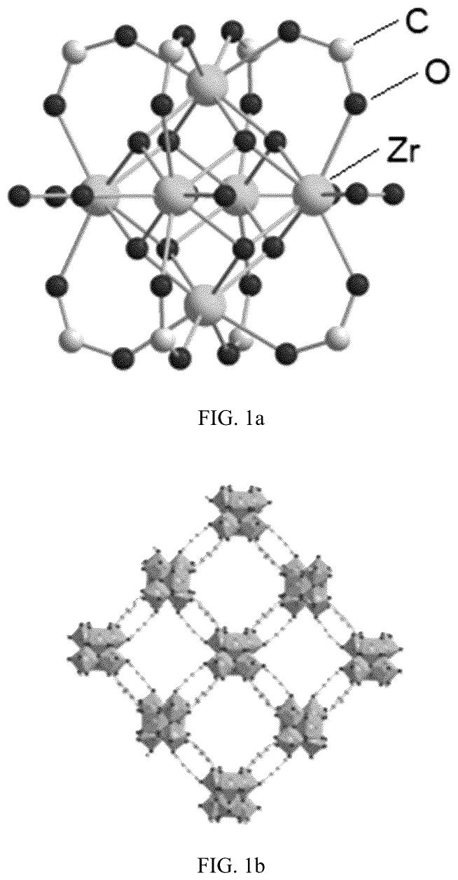

### DETAILED DESCRIPTION OF PREFERRED EMBODIMENTS

In order to have a clearer understanding of the technical features, purposes and beneficial effects of the present disclosure, the technical solutions of the present disclosure are described in detail as follows, but are not to be construed as limiting the implementable scope of the present disclosure.

### Example 1

This example provides a method for preparing a zirconium-based metal-organic framework material and testing of the adsorption separation performance of alkane isomers, specifically as follows.

0.17 mmol of zirconium chloride and 0.06 mmol of dpetc were added to a mixture solution of 6 mL of formic acid and 4 ml of N,N-dimethylformamide, stirred for 30 min, and transferred to a 20 mL glass vial. The vial was capped tightly and reacted in an oven at 120° C. for 72 h. After cooling and filtration, a white powder crystal A was obtained.

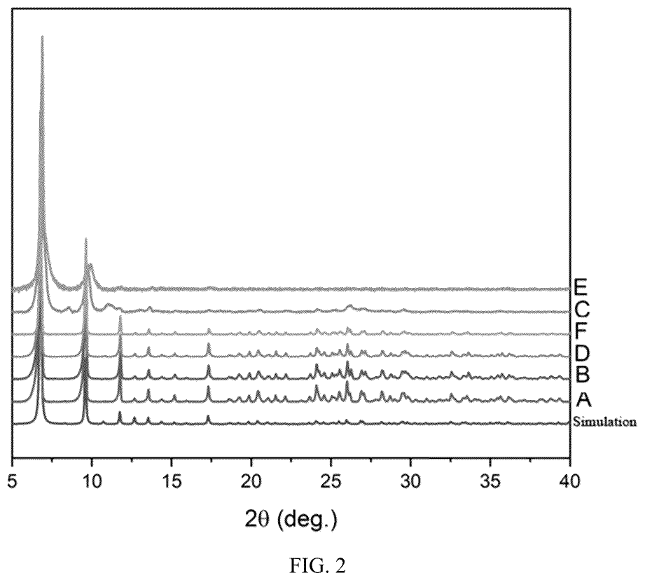

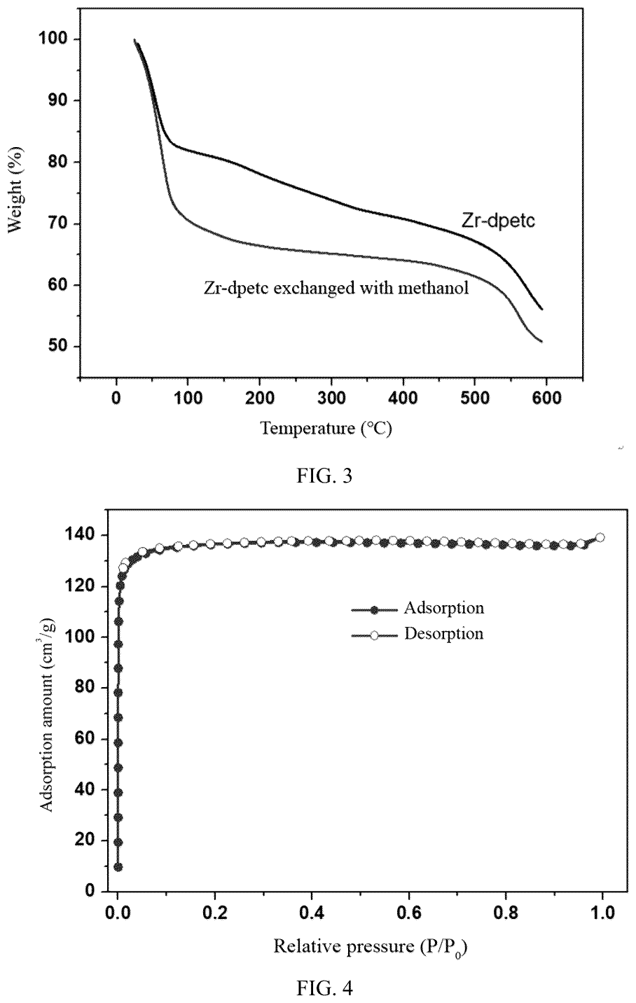

The material obtained by filtration was immersed in methanol solution for 48 h to allow the methanol with a low boiling point to fully replace N,N-dimethylformamide solvent with a high boiling point inside the channels of the material, and then the solvent-exchanged material was filtered to obtain a material B. The X-ray diffraction patterns of the white powder crystal A and the material B are shown in FIG. 2, and the thermogravimetric curves of them are shown in FIG. 3.

In order to test the size of specific surface area of the synthesized adsorbent, the material B was vacuum degassed at 120° C. for 12 h to obtain the zirconium-based metal-organic framework material C having removed the solvent in the channels, which was subjected to a test for nitrogen adsorption-desorption isotherm at 77 K. The results are shown in FIG. 4, and the specific surface area of the material C was tested to be 630 m²/g.

In order to test the stability of the adsorbent materials, the adsorbent B was placed respectively in an oven at 120° C., in water at a temperature of 80° C., and in air at a humidity of 90%, and all of them were left for 7 days to obtain the materials (D, E, and F) for X-ray diffraction analysis tests. As shown in FIG. 2, the test results show that D, E and F still maintain the intact crystal structure, indicating a good stability.

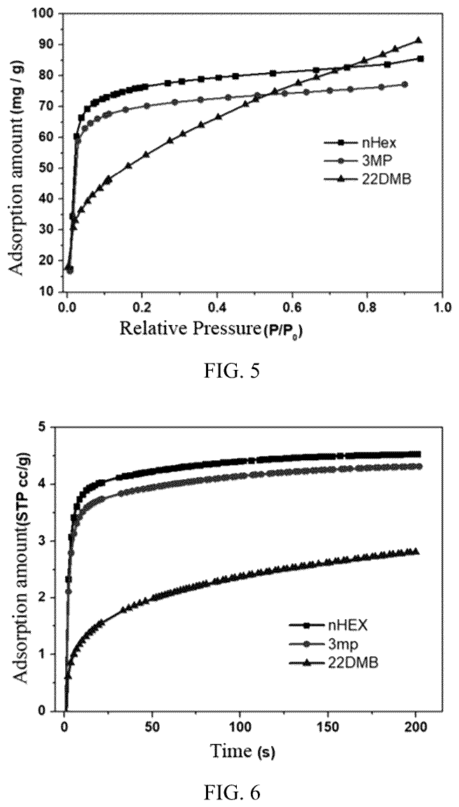

In order to test the adsorption separation performance of the synthesized adsorbent, the desorption-treated adsorbent C was tested for single-component adsorption isotherms of n-hexane, 3-methylpentane, and 2,2-dimethylbutane, respectively. As shown in FIG. 5, under the test conditions of a temperature of 30° C. and a pressure of 0.9 bar, the adsorption amount of n-hexane was 85 mg/g, the adsorption amount of 3-methylpentane was 72 mg/g, and the adsorption amount of 2,2-dimethylbutane was 86 mg/g.

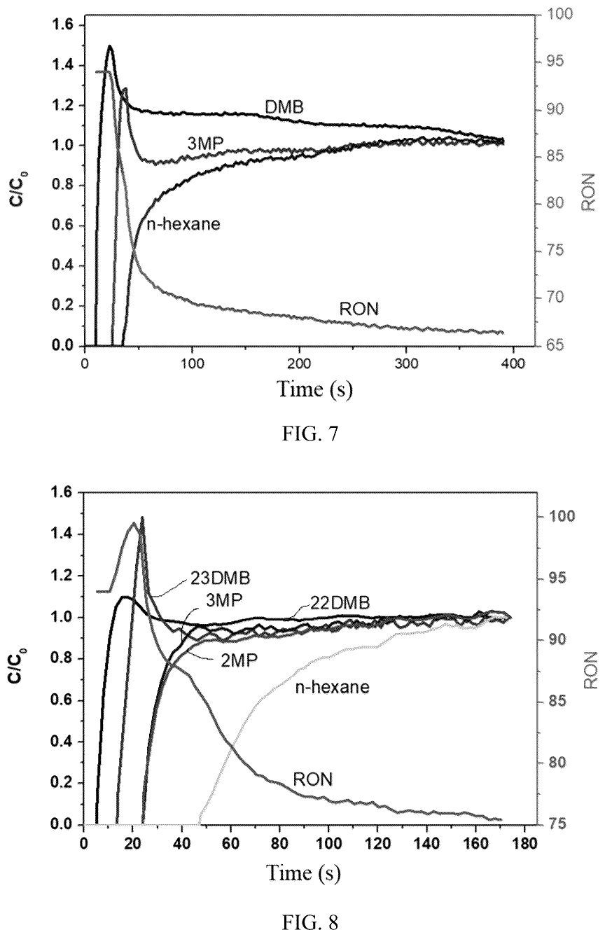

A ternary mixture of equimolar n-hexane, 3-methylpentane and 2,2-dimethylbutane was passed into an adsorbent-filled adsorbent column using helium as the carrier gas. As shown in FIG. 6, at a temperature of 30° C., a pressure of 1 bar, and a mixture gas flow rate of 1 mL/min, breakthrough of 2,2-dimethylbutane began at the 10th minute, breakthrough of 3-methylpentane began at the 25th minute, and breakthrough of n-hexane began at the 35th minute. As shown in FIG. 7, a gasoline blending component having an octane number of 95 or more can be obtained by the present method.

A quinary mixture of equimolar n-hexane, 2-methylpentane, 3-methylpentane, 2,3-dimethylbutane and 2,2-dimethylbutane was passed into an adsorbent column filled with material C using helium as the carrier gas, at a temperature of 30° C., a pressure of 1 bar, and a mixture gas flow rate of 1 mL/min. Upon testing, as shown in FIG. 8, breakthrough of 2,2-dimethylbutane occurred at the 5th minute, breakthrough of 2,3-dimethylbutane occurred at the 13th minute, and breakthrough of 2-methylpentane and 3-methylpentane occurred at the 24th minute. The fractions prior to breakthrough of monomethyl components were collected and condensed to obtain a product with an octane number of up to 95. Breakthrough of n-hexane occurred at the 47th minute, and the fractions prior to breakthrough of n-hexane were collected and condensed to obtain mono-branched alkanes. After the breakthrough of n-hexane, the venting was stopped and the adsorption column was heated to 150° C. for desorption, and a product with a n-hexane content greater than 80% was collected.

### Example 2

This example provides a method for preparing a zirconium-based metal-organic framework material and testing of the adsorption separation performance of alkane isomers, specifically as follows.

0.35 mmol of zirconium chloride and 0.12 mmol of dpetc were added to a mixture solution of 3 mL of acetic acid and 2 mL of N,N-dimethylacetamide (DMA), treated by ultrasonication for 30 min, and transferred to a 10 mL glass tube of a microwave synthesizer. The glass tube was capped tightly, and then placed in the microwave synthesizer and heated at 100° C. for 5 min. After cooling and filtration, a white powder was obtained. The white powder was immersed in dichloromethane solution for 48 h and filtered to obtain an exchanged sample, which was then vacuum degassed at 80° C. for 15 h to obtain a desorption sample. A mixture gas of n-hexane, 2-methylpentane, 3-methylpentane, 2,3-dimethylbutane, 2,2-dimethylbutane and oxygen in a molar ratio of 19:19:19:19:19:5 was passed into an adsorption column filled with the desorption sample. At a temperature of 30° C., a pressure of 0.5 bar, and a mixture gas flow rate of 2 mL/min, breakthrough of oxygen occurred first, followed by breakthrough of 2,2-dimethylbutane and 2,3-dimethylbutane, and a product with an octane number of 96 was obtained by condensation. Subsequently, breakthrough of 2-methylpentane and 3-methylpentane occurred, and finally breakthrough of n-hexane occurred. After breakthrough, the venting was stopped and the adsorption column was purged using helium. After condensation of the purge gas, n-hexane with a purity of 85% was obtained.

### Example 3

This example provides a method for preparing a zirconium-based metal-organic framework material and testing of the adsorption separation performance of alkane isomers, specifically as follows.

0.35 mmol of aluminum zirconium oxide and 0.40 mmol of dpetc were added to a mixture solution of 3 mL of acetic acid and 2 mL of N,N-dimethylacetamide (DMA), treated by ultrasonication for 30 min, and transferred to a 25 mL stainless steel reactor with a Teflon liner. The reactor was capped tightly, and then placed in an oven at 150° C. for 24 h. After cooling and filtration, a white powder A was obtained. The white powder was immersed in a solution of n-hexane for 48 h, and filtered to obtain a material B, which was then vacuum degassed at 150° C. for 8 h to obtain a material C. A mixture gas of n-hexane, 2-methylpentane, 2,3-dimethylbutane, 2,2-dimethylbutane, nitrogen and helium in a molar ratio of 20:20:20:20:5:5 was passed into an adsorption column filled with the desorption sample. At a temperature of 40° C., a pressure of 1 bar, and a mixture gas flow rate of 4 mL/min, breakthrough of nitrogen and helium occurred first, followed by breakthrough of 2,2-dimethylbutane and 2,3-dimethylbutane, and a product with an octane number of 95 was obtained by condensation. Subsequently, breakthrough of 2-methylpentane occurred, and finally breakthrough of n-hexane occurred. After breakthrough, the venting was stopped and the adsorption column was purged using helium. After condensation of the purge gas, n-hexane with a purity of 82% was obtained.

### Example 4

This example provides a method for preparing a zirconium-based metal-organic framework material and testing of the adsorption separation performance of alkane isomers, specifically as follows.

0.35 mmol of zirconium sulfate and 0.12 mmol of dpetc were added to a mixture solution of 3 mL of acetic acid and 10 mL of N,N-dimethylacetamide (DMA), treated by ultrasonication for 30 min, and transferred to a 10 mL glass tube of a microwave synthesizer. The glass tube was capped tightly, and then placed in the microwave synthesizer and heated at 160° C. for 50 min. After cooling and filtration, a white powder was obtained. The white powder was immersed in ethanol solution for 48 h and filtered to obtain a filtered sample, which was then vacuum degassed at 200° C. for 6 h to obtain a desorption sample. A quinary mixture of equimolar n-hexane, 2-methylpentane, 3-methylpentane, 2,3-dimethylbutane and 2,2-dimethylbutane was passed into an adsorption column filled with the desorption sample using helium as the carrier gas. At a temperature of 100° C., a pressure of 1 bar, and a mixture gas flow rate of 2 mL/min, breakthrough of 2,2-dimethylbutane and 2,3-dimethylbutane occurred first, and a product with an octane number of 97 was obtained by condensation. Subsequently, breakthrough of 2-methylpentane and 3-methylpentane occurred, and finally breakthrough of n-hexane occurred. After breakthrough, the venting was stopped and the adsorption column was purged using helium. After condensation of the purge gas, n-hexane with a purity of 86% was obtained.

### Example 5

This example provides a method for preparing a zirconium-based metal-organic framework material and testing of the adsorption separation performance of alkane isomers, specifically as follows.

0.35 mmol of zirconium chloride and 0.12 mmol of dpetc were added to a mixture solution of 8 mL of benzoic acid and 2 mL of N,N-dimethylacetamide (DMA), treated by ultrasonication for 30 min, and transferred to a 25 mL stainless steel reactor with a Teflon liner. The reactor was capped tightly, and then placed in an oven at 200° C. for 160 h. After cooling and filtration, a white powder was obtained. The white powder was immersed in ethanol solution for 48 h and filtered to obtain a filtered sample, which was then vacuum degassed at 160° C. for 8 h to obtain a desorption sample. A quinary mixture liquid of equimolar n-hexane, 2-methylpentane, 3-methylpentane, 2,3-dimethylbutane and 2,2-dimethylbutane was passed into an adsorption column filled with the desorption sample. At a temperature of 30° C., a pressure of 5 bar, and a mixture gas flow rate of 1 mL/min, breakthrough of 2,2-dimethylbutane and 2,3-dimethylbutane occurred first, and a product with an octane number of 96 was obtained by condensation. Subsequently, breakthrough of 2-methylpentane and 3-methylpentane occurred, and finally breakthrough of n-hexane occurred. After breakthrough, the venting was stopped and the adsorption column was heated to 200° C. and purged using helium. After condensation of the purge gas, n-hexane with a purity of 82% was obtained.

### Comparative Example 1

This comparative example provides a method for separating hexane isomers by adsorption using 5A molecular sieve, specifically as follows.

A quinary mixture of equimolar n-hexane, 2-methylpentane, 3-methylpentane, 2,3-dimethylbutane and 2,2-dimethylbutane was passed into an adsorption column filled with activated 5A molecular sieve using helium as the carrier gas. At a temperature of 100° C., a pressure of 1 bar, and a mixture gas flow rate of 2 mL/min, breakthrough of 2,2-dimethylbutane, 2,3-dimethylbutane, 2-methylpentane and 3-methylpentane occurred first, and a product with an octane number of 88 was obtained by condensation. Finally, breakthrough of n-hexane occurred. After breakthrough, the venting was stopped and the adsorption column was purged using helium. After condensation of the purge gas, n-hexane with a purity of 85% was obtained. The 5A molecular sieve is a solid adsorbent having one-dimensional channels with a diameter of about 5 Å. The diameter of channels is between the kinetic diameters of straight hexane and branched hexane, which, together with the more rigid structural characteristics of the molecular sieve, only allow the 5A molecular sieve to adsorb the straight alkane isomer, rather than all of the branched isomers. Therefore, the 5A molecular sieve can only separate straight and branched hexane isomers, but cannot separate mono-branched and di-branched hexane isomers.

### Comparative Example 2

This comparative example provides an adsorption separation method for hexane isomers using a UiO-66 (Zr) metal-organic framework material, wherein UiO-66 (Zr) has a three-dimensional channel structure formed by coordination of metal Zr and terephthalic acid, and contains octahedral cages with a diameter of 1.1 nm and tetrahedral cages with a diameter of 0.8 nm, with a pore window of about 0.5-0.7 nm. The adsorption separation method is specified as follows:

A quinary mixture of equimolar n-hexane, 2-methylpentane, 3-methylpentane, 2,3-dimethylbutane and 2,2-dimethylbutane was passed into an adsorption column filled with activated UiO-66 using helium as the carrier gas. At a temperature of 100° C., a pressure of 1 bar, and a mixture gas flow rate of 2 mL/min, breakthrough of n-hexane, 2-methylpentane and 3-methylpentane occurred first, and a product with an octane number of 56 was obtained by condensation. Subsequently, breakthrough of 2,3-dimethylbutane and 2,2-dimethylbutane occurred. After breakthrough, the venting was stopped and the adsorption column was purged using helium. After condensation of the purge gas, a mixture containing 2,2-dimethylbutane and 2,3-dimethylbutane was obtained, which also included the remaining n-hexane, 2-methylpentane and 3-methylpentane. The two alkanes, 2,2-dimethylbutane and 2,3-dimethylbutane, together comprise 52% of the total amount of the mixture, and the octane number of the mixture is 74. The separation method of the comparative example cannot obtain a product with a high octane number.

The foregoing description is specific embodiments of the present disclosure. It should be noted that for the person of ordinary skill in the art, several improvements and modifications can be made without departing from the principles of the present disclosure, and these improvements and modifications are also considered as within the protection scope of the present disclosure.

1. A zirconium-based metal-organic framework material having a chemical structural formula [C₁₈H₆O₁₆Zr₃]ₙ, wherein the zirconium-based metal-organic framework material comprises a zirconium element and an organic ligand which forms a coordination bond with the zirconium element, and the organic ligand is diphenylethyne-3,3′,5,5′-tetracarboxylic acid.

2. The zirconium-based metal-organic framework material according to claim 1, wherein the crystal of the zirconium-based metal-organic framework material belongs to the tetragonal crystal system with 14/mmm space group.

3. The zirconium-based metal-organic framework material according to claim 1, wherein the molecular structure of the zirconium-based metal-organic framework material is a three-dimensional network structure with one-dimensional channels.

4. The zirconium-based metal-organic framework material according to claim 3, wherein the one-dimensional channels have a size of 5-7 Å.

5. The zirconium-based metal-organic framework material according to claim 3, wherein the three-dimensional network structure is formed by ZrO₆ octahedron connected with the organic ligand.

6. The zirconium-based metal-organic framework material according to claim 5, wherein the ZrO₆ octahedron comprises six Zr atoms.

7. The zirconium-based metal-organic framework material according to claim 1, wherein the zirconium-based metal-organic framework material has a specific surface area of 500-1000 m²/g.

8. The zirconium-based metal-organic framework material according to claim 7, wherein the zirconium-based metal-organic framework material has a thermal decomposition temperature of 350-500° C.

9. The zirconium-based metal-organic framework material according to claim 7, wherein the zirconium-based metal-organic framework material is in the form of white powder crystals.

10. A method for preparing the zirconium-based metal-organic framework material according to claim 1, comprising the steps of:
(1) mixing a zirconium salt, an organic ligand, a first solvent and an acid in a proportion, and performing a solvothermal reaction or a microwave synthesis process to obtain a semi-finished product; and
(2) removing the solvent in the channels of the semi-finished product, to obtain a finished product of the zirconium-based metal-organic framework material.

11. The method according to claim 10, wherein the molar ratio of the zirconium salt, the organic ligand, the first solvent and the acid is 10: (1-100): (1-100): (2-200).

12. The method according to claim 10, wherein the zirconium salt is selected from at least one of zirconium nitrate, zirconium chloride, aluminum zirconium oxide, and zirconium sulfate;
the organic ligand is diphenylethyne-3,3′,5,5′-tetracarboxylic acid;
the first solvent is selected from at least one of N,N-dimethylformamide, N,N-dimethylacetamide, and N,N-diethylformamide
the acid is selected from at least one of formic acid, acetic acid, hydrochloric acid, and benzoic acid.

13. (canceled)

14. (canceled)

15. (canceled)

16. The method according to claim 10, wherein the reaction temperature of the solvothermal reaction is 80-200° C. and the reaction time is 12-72 h;
the reaction temperature of the microwave synthesis process is 80-180° C. and the reaction time is 1-60 min;
the removing the solvent in the channels of the semi-finished product is carried out by vacuum drying the semi-finished product, or by impregnating the semi-finished product in a second solvent for solvent exchange, and then vacuum drying; the second solvent is selected from at least one of methanol, dichloromethane, ethanol, and acetone.

17. (canceled)

18. (canceled)

19. (canceled)

20. (canceled)

21. (canceled)

22. (canceled)

23. (canceled)

24. (canceled)

25. (canceled)

26. (canceled)

27. An adsorption separation method for alkane isomers, comprising: passing a mixture gas or mixture liquid containing hexane isomers through an adsorption column filled with an adsorbent, collecting the constituent isomers one by one, and desorbing the adsorbent after completion of the collection; wherein the adsorbent is the zirconium-based metal-organic framework material according to claim 1.

28. The adsorption separation method for alkane isomers according to claim 27, wherein the desorption is carried out by one or more of heating, vacuum treatment, and inert gas purging.

29. The adsorption separation method for alkane isomers according to claim 27, wherein the adsorption temperature is 0-200° C.

30. The adsorption separation method for alkane isomers according to claim 27, wherein the total pressure of the mixture gas during the adsorption is 0-5 bar.

31. The adsorption separation method for alkane isomers according to claim 27, wherein the desorption temperature is 100-200° C.

32. The adsorption separation method for alkane isomers according to claim 27, wherein the total pressure of the mixture gas during the desorption is 0.05-1 bar.

33. The adsorption separation method for alkane isomers according to claim 27, wherein the total amount of the hexane isomers is 70-90% of the total mass of the mixture gas or mixture liquid.
